(19) United States
(12) Patent Application Publication
(10) Pub. No.: US 2009/0143451 A1
(43) Pub. Date: Jun. 4, 2009

(54) COMPOUNDS THAT INCREASE TELOMERASE REVERSE TRANSCRIPTASE (TERT) EXPRESSION AND METHODS FOR USING THE SAME

(76) Inventors: William H. Andrews, Reno, NV (US); Laura A. Briggs, Reno, NV (US); Christopher A. Foster, Carmichael, CA (US); Lancer K. Brown, Sparks, NV (US); Mieczyslaw A. Piatyszek, Morgan Hill, CA (US); Federico C.A. Gaeta, Mountain View, CA (US); Munirathnam Chaguturu, Lawrence, KS (US); Jian Zhang, Fremont, CA (US); Tom Kerley, Gardnerville, NV (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/270,552

(22) Filed: Nov. 13, 2008

Related U.S. Application Data
(60) Provisional application No. 60/988,043, filed on Nov. 14, 2007.

(51) Int. Cl. A61K 31/422; C12N 5/02; C12Q 1/02

(52) U.S. Cl. 514/378; 435/375; 435/29

(57) ABSTRACT

Compounds, and methods of using the same, are provided which increase the expression of telomerase reverse transcriptase (TERT) in a cell. These compounds and methods find use in a variety of applications in which increased expression of telomerase is desired, including immortalization of cell lines and treating conditions in a subject characterized by cellular senescence.

### CROSS-REFERENCE TO RELATED APPLICATIONS

Pursuant to 35 U.S.C. § 119 (e), this application claims priority to the filing date of U.S. Provisional Patent Application Ser. No. 60/998,043 filed Nov. 14, 2007; the disclosure of which application is herein incorporated by reference.

### INTRODUCTION

Telomeres, which define the ends of chromosomes, consist of short, tandemly repeated DNA sequences loosely conserved in eukaryotes. For example, human telomeres consist of many kilobases of (TTAGGG)n together with various associated proteins. Small amounts of these terminal sequences or telomeric DNA are lost from the tips of the chromosomes during S phase because of incomplete DNA replication. Many human cells progressively lose terminal sequence with cell division, a loss that correlates with the apparent absence of telomerase in these cells. The resulting telomeric shortening has been demonstrated to limit cellular lifespan.

Telomerase is a ribonucleoprotein that synthesizes telomeric DNA. In general, telomerase is made up of two components: (1) an essential structural RNA (TR or TER) (where the human component is referred to in the art as hTR or hTER); and (2) a catalytic protein (telomerase reverse transcriptase or TERT) (where the human component is referred to in the art as hTERT). Telomerase works by recognizing the 3′ end of DNA, e.g., telomeres, and adding multiple telomeric repeats to its 3′ end with the catalytic protein component, e.g., hTERT, which has polymerase activity, and hTER which serves as the template for nucleotide incorporation. Of these two components of the telomerase enzyme, both the catalytic protein component and the RNA template component are activity-limiting components.

Because of its role in cellular senescence and immortalization, there is much interest in the identification of compounds that regulate telomerase activity.

### SUMMARY

Compounds, and methods of using the same, are provided which increase the expression of telomerase reverse transcriptase (TERT). These compounds and methods find use in a variety of applications in which increased expression of telomerase is desired, including immortalization of cell lines and treating conditions in a subject characterized by cellular senescence.

### DEFINITIONS

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope.

“Acyl” refers to a radical —C(O)R, where R is hydrogen, alkyl, alkenyl, cycloalkyl, heterocycloalkyl, aryl, arylalkyl, heteroalkyl, heteroalkenyl, or heteroaryl as defined herein. Representative examples include, but are not limited to, formyl, acetyl, cylcohexylcarbonyl, cyclohexylmethylcarbonyl, benzoyl, benzylcarbonyl and the like.

“Acylamino” refers to a radical —NR′C(O)R, where R′ is hydrogen, alkyl, cycloalkyl, heterocycloalkyl, aryl, arylalkyl, heteroalkyl, heteroaryl, heteroarylalkyl and R is hydrogen, alkyl, alkoxy, cycloalkyl, heterocycloalkyl, aryl, arylalkyl, heteroalkyl, heteroaryl or heteroarylalkyl, as defined herein. Representative examples include, but are not limited to, formylamino, acetylamino, cyclohexylcarbonylamino, cyclohexylmethyl-carbonylamino, benzoylamino, benzylcarbonylamino and the like.

“Acyloxy” refers to the group —OC(O)H, —OC(O)-alkyl, —OC(O)-aryl or —OC(O)-cycloalkyl.

“Aliphatic” refers to hydrocarbyl organic compounds or groups characterized by a straight, branched or cyclic arrangement of the constituent carbon atoms and an absence of aromatic unsaturation. Aliphatics include, without limitation, alkyl, alkylene, alkenyl, alkynyl and alkynylene. Lower aliphatic groups typically have from 1 or 2 to 6 or 12 carbon atoms.

“Alkenyl” refers to monovalent olefinically unsaturated hydrocarbyl groups having up to about 11 carbon atoms, particularly, from 2 to 8 carbon atoms, and more particularly, from 2 to 6 carbon atoms, which can be straight-chained or branched and having at least 1 and particularly from 1 to 2 sites of olefinic unsaturation. Particular alkenyl groups include ethenyl (—CH═CH₂), n-propenyl (—CH₂CH═CH₂), isopropenyl (—C(CH₃)═CH₂), vinyl and substituted vinyl, and the like.

“Alkoxy” refers to the group —O-alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Alkoxycarbonyl” refers to a radical —C(O)-alkoxy where alkoxy is as defined herein.

“Alkoxycarbonylamino” refers to the group —NRC(O)OR′ where R is hydrogen, alkyl, aryl or cycloalkyl, and R′ is alkyl or cycloalkyl.

“Alkyl” refers to monovalent saturated aliphatic hydrocarbyl groups particularly having up to about 12 or 18 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “alkyl” also includes “cycloalkyls” as defined herein.

“Alkylene” refers to divalent saturated aliphatic hydrocarbyl groups particularly having up to about 12 or 18 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkynyl” refers to acetylenically unsaturated hydrocarbyl groups particularly having up to about 12 or 18 carbon atoms and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

“Amino” refers to the radical —NH₂.

“Aminocarbonyl” refers to the group —C(O)NRR where each R is independently hydrogen, alkyl, aryl or cycloalkyl, or where the R groups are joined to form an alkylene group.

“Aminocarbonylamino” refers to the group —NRC(O)NRR where each R is independently hydrogen, alkyl, aryl or cycloalkyl, or where two R groups are joined to form an alkylene group.

“Aminocarbonyloxy” refers to the group —OC(O)NRR where each R is independently hydrogen, alkyl, aryl or cycloalkyl, or where the R groups are joined to form an alkylene group.

“Aralkyl” or “arylalkyl” refers to an alkyl group, as defined above, substituted with one or more aryl groups, as defined above.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms.

“Aryloxy” refers to —O-aryl groups wherein “aryl” is as defined herein.

“Azido” refers to the radical —N₃.

“Carboxyl” refers to the radical —C(O)OH.

“Cyano” refers to the radical —CN.

“Cycloalkenyl” refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

“Heterocycloalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl.

“Halogen” refers to fluoro, chloro, bromo and iodo.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. heterocycloalkyl, aryl, e.g. heteroaryl, cycloalkenyl, e.g., heterocycloalkenyl, cycloheteroalkenyl, e.g., heterocycloheteroalkenyl and the like having from 1 to 5, and particularly from 1 to 3 heteroatoms. A heteroatom is any atom other than carbon or hydrogen and is typically, but not exclusively, nitrogen, oxygen, sulfur, phosphorus, boron, chlorine, bromine, or iodine. An unsubstituted heteroatom refers to a pendant heteroatom such as an amine, hydroxyl and thiol. A substituted heteroatom refers to a heteroatom that is other than a pendant heteroatom.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. The heteroaryl group can be a 5-20 membered heteroaryl, or 5-10 membered heteroaryl. Particular heteroaryl groups are those derived from thiophen, pyrrole, benzothiophene, benzofuran, indole, pyridine, quinoline, imidazole, oxazole and pyrazine.

“Heterocycle” refers to organic compounds that contain a ring structure containing atoms in addition to carbon, such as sulfur, oxygen or nitrogen, as part of the ring. They may be either simple aromatic rings or non-aromatic rings. Typical examples are azoles, morpholine, piperazine, pyridine, pyrimidine and dioxane.

“Hydroxyl” refers to the radical —OH.

“Nitro” refers to the radical —NO₂.

“Stereoisomer” as it relates to a given compound is well understood in the art, and refers to another compound having the same molecular formula, wherein the atoms making up the other compound differ in the way they are oriented in space, but wherein the atoms in the other compound are like the atoms in the given compound with respect to which atoms are joined to which other atoms (e.g. an enantiomer, a diastereomer, or a geometric isomer). See for example, Morrison and Boyd, Organic Chemistry, 1983, 4th ed., Allyn and Bacon, Inc., Boston, Mass., p. 123.

“Substituted” refers to a group in which one or more hydrogen atoms are each independently replaced with the same or different substituent(s). “Substituted” groups particularly refer to groups having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂. Typical substituents include, but are not limited to, —X, —R⁸ (with the provisio that R⁸ is not hydrogen), —O—, ═O, —OR⁸, —SR⁸, —S⁻, ═S, —NR⁸R⁹, ═NR⁸, —CX₃, —CF₃, —CN, —OCN, —SCN, —NO, —NO₂, ═N₂, —N₃, —S(O)₂O⁻, —S(O)₂OH, —S(O)₂R⁸, OS(O₂)O⁻, —OS(O)₂R⁸, —P(O) (O—)₂, —P(O)(OR⁸)(O⁻), —OP(O)(OR⁸)(OR⁹), —C(O)R⁸, —C(S)R⁸, —C(O)OR⁸, —C(O)NR⁸R⁹, —C(O)O⁻, —C(S)OR⁸, —NR¹⁰C(O)NR⁸R⁸R⁹, —NR¹⁰C(S)NR⁸R⁹, —NR¹¹C(NR¹⁰)NR⁸R⁹ and —C(NR¹⁰)NR⁸R⁹, where each X is independently a halogen.

“Substituted acyl” includes those groups recited in the definition of “substituted” herein, and particularly refers to the group —C(O)R where R selected from the group consisting of hydrogen, alkyl, substituted alkyl, alkenyl, substituted alkenyl, alkynyl, substituted alkynyl, amino, substituted amino, aryl, arylalkyl, cycloalkyl, substituted cycloalkyl, heterocycloalkyl, heteroalkyl, or heteroaryl as defined herein.

“Substituted amino” includes those groups recited in the definition of “substituted” herein, and particularly refers to the group —N(R)₂ where each R is independently selected from the group consisting of hydrogen, alkyl, substituted alkyl, alkenyl, substituted alkenyl, alkynyl, substituted alkynyl, aryl, cycloalkyl, substituted cycloalkyl, and where both R groups are joined to form an alkylene group.

“Thioalkoxy” refers to the group —S-alkyl.

“Thioaryloxy” refers to the group —S-aryl.

“Thioketo” refers to the group ═S.

“Thiol” refers to the group —SH.

One having ordinary skill in the art will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

### DETAILED DESCRIPTION

As mentioned above, compounds are provided that increase the expression of TERT in a cell. The subject compounds find use in a variety of research and therapeutic applications. The compounds include substituted isoxazoles. In certain embodiments, the substituted isoxazoles act by inhibiting binding of a transcriptional repressor protein or protein complex to at least one Site C binding site of a TERT expression system. The methods include increasing TERT expression in a cell containing a TERT expression system that comprises at least one Site C binding site in its promoter, by contacting the cell with an effective amount of a substituted isoxazole compound of the invention. Also included is a method of screening a compound for inhibiting binding of a transcriptional repressor protein/protein complex to a TERT promoter that comprises at least one Site C binding site, where the method comprises determining whether a substituted azole compound is capable of inhibiting binding of the transcriptional repressor protein/protein complex to the Site C binding site.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, the function and structure of the compounds of the invention are described first in greater detail, followed by a description of applications in which the compounds finds use.

### Functional Properties

The compounds of the invention increase TERT expression in a cell. The cells in which the compounds of the invention exhibit activity are ones that include a TERT gene containing a Site C site in its promoter region, e.g., in the TERT gene minimal promoter. By increasing TERT expression is meant that the expression level of the TERT encoding mRNA is increased by 2-fold or more, such as by 5-fold or more and sometimes by 25-, about 50-, about 100-fold or more and in certain embodiments 300-fold or more or higher, as compared to a control, i.e., expression in the same cell not contacted with the compound of interest (e.g., by using the assay described in Published United States Patent Application Publication No. US-2006-0199171-A1, the disclosure of which assay is herein incorporated by reference). Alternatively, in cases where expression of the TERT gene in a cell is so low that it is undetectable, the expression level of the TERT encoding mRNA is considered to be increased if expression is increased to a level that is easily detectable, e.g., by using the assay described in Published United States Patent Application Publication No. US-2006-0199171-A1, the disclosure of which assay is herein incorporated by reference.

In certain embodiments, the target cell in which TERT expression is increased is a normal cell, e.g., a somatic cell. In some of these embodiments, the compounds of the invention are used to increase the proliferative capacity of a cell. The term “proliferative capacity” as used herein refers to the number of divisions that a cell can undergo, and preferably to the ability of the target cell to continue to divide where the daughter cells of such divisions are not transformed, i.e., they maintain normal response to growth and cell cycle regulation. As such, the compounds of the invention find use is in the delay of the occurrence of cellular senescence. The compounds of the invention may delay the onset of cellular senescence by a factor of 1.2 or more, such as 2 fold or more, including 5 fold or more where in certain embodiments the delay is even greater, e.g., 10-, 20-, 50-fold or more or even higher, compared to a control.

In certain embodiments, the compounds of the invention modulate the interaction of a transcriptional repressor complex and a Site C site in the TERT promoter. By transcriptional repressor complex is meant a complex containing at least one factor (e.g., protein), wherein the complex binds specifically to a Site C site in the TERT promoter. For example, the transcriptional repressor complex can be a single protein that binds specifically to the Site C site in the TERT promoter (or minimal promoter). In contrast, the transcriptional repressor complex can contain a number of factors (e.g., proteins) that together bind specifically to the Site C site in the TERT promoter. In general, binding of the transcriptional repressor complex to a Site C site in the TERT promoter represses or reduces transcription of the TERT gene.

In certain embodiments, modulating the interaction of a transcriptional repressor complex and a Site C site means that the interaction is inhibited or reduced. In certain of these embodiments, the mechanism of activity of the compounds is by specific, direct interaction with the transcriptional repressor protein complex thereby preventing its binding to Site C in the TERT promoter. In certain embodiments, the binding of the compound to the transcriptional repressor complex competitively inhibits Site C DNA binding (meaning that the compound binds to the DNA-binding site of the transcriptional repressor complex) while in other embodiments the compound allosterically inhibits Site C DNA binding of the transcriptional repressor (meaning that it binds to a site other than to the DNA binding site of the transcriptional repressor). In certain embodiments, the compound binds to a member of the transcriptional repressor complex other than the DNA binding subunit to exert its inhibitory activity.

In certain embodiments, the compounds of the present invention reduce the repressive activity of a TERT transcriptional repressor complex of one or more factors (e.g., proteins), e.g., by inhibiting the binding of a transcriptional repressor to its cognate DNA binding site in the TERT minimal promoter. Of particular interest is the Site C DNA binding site within the −66 to −51 region of the TERT minimal promoter. This repressor site has been described in U.S. Pat. No. 6,686,159, which is incorporated herein by reference. In certain embodiments, the Site C sequence is:

(SEQ ID NO:1)

GGCCCCGCCCTCTCCTCGCGGCGCGAGTTTCAGGCAGCGCT

In certain embodiments, the target Site C sequence is a portion or subsequence of the above sequence, such as:

| | |
|---|---|
| GGCGCGAGTTTCA; | (SEQ ID NO:2) |
| CGCGAGTTTC; | (SEQ ID NO:3) |
| or | |
| GGCGCGAGTTTCAGGCAGCGC. | (SEQ ID NO:4) |

Site C-binding transcriptional repressor complexes are described in U.S. patent application Ser. No. 11/088,001 filed on Mar. 22, 2005 entitled “Methods and Compositions for Modulating Telomerase Reverse Transcriptase (TERT) Expression”, which is incorporated by reference herein in its entirety. As described therein, transcriptional repressor complexes that bind to Site C site include any known or later discovered members of LSF family including any homolog or any protein or polypeptide with at least 50%, 70%, or 90% of its amino acids identical to a member of LSF family, especially within its functional regions, e.g., its DNA binding domain or regions involved in protein-protein interaction. In general, LSF family is a family of proteins related to mammalian transcription factor LSF. Members of LSF family usually include LBP1a, LBP1b, LBP1c, LBP1d, LBP9, LBP32v1, LBP32v2, SOMv1, SOMv2, SOMv3, and BOM. LBP1d is a splice variant of LBP1c while LBP1a is a splice variant of LBP1b. In addition, members the LSF family also include a splice variant of LBP1c, called LBP1c2, and a variant of BOM, called BOMv2, as well as any protein or polypeptide capable of binding to or interacting with one or more members related to LSF, e.g., YY1, NF-E4, Fe65, APP-CT, NFPB, and SP1.

### Structural Features

The compounds of the invention may include one or more aromatic or heteroaromatic rings. The compounds may include from 5 to 30 carbon atoms, such as 7 to 25 carbon atoms, e.g., 10 to 15 carbon atoms. In certain embodiments, the compounds are substituted azoles, e.g., substituted with heterocyclic and acyl substituents. In some embodiments, the azole, heterocycle and acyl substituents are in conjugation with each other. The azole compounds of the invention may include an aromatic heterocycle with two or more hetero atoms selected from nitrogen, oxygen and sulfur. Examples include azole compounds such as pyrazoles, imidazoles, triazoles, tetrazoles, thiazoles, isothiazoles, oxazoles, and isoxazoles. In some embodiments, the substituted azole is a substituted isoxazole. Substituent bonds to the azole may be to the carbons of the heterocyclic ring. In another embodiment, the bonds may be to the 3- and 5-positions of the azole core structure.

In certain embodiments, the heterocyclic substituent includes a five-membered aromatic heterocycle. The heterocycle includes at least one oxygen or sulfur atom. In some embodiments, the heterocyclic substituent comprises a thiophene group. In some embodiments, bonds to the heterocycle are made to the 2-position of the heterocycle.

In some embodiments, the acyl substituent comprises an amide, where the amide may be an alkyl amide. In certain embodiments, the alkyl amide substituent is a propylamide. In certain embodiments, the acyl substituent and said azole core may be to the carbonyl carbon of the acyl substituent.

Thus in certain embodiments, the compounds of the invention comprise a substituted azole, such as a substituted azole having a structure of formula (I):

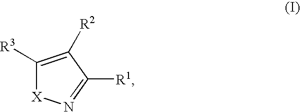

where each R¹, R² and R³ is the same or different and each independently comprises hydrogen or a residue of a group selected from a substituted or unsubstituted acyl, a substituted or unsubstituted amino, a substituted or unsubstituted heterocyle, a substituted or unsubstituted straight-chain or branched C₁-C₂₀-alkyl or C₂-C₂₀-alkenyl (with or without asymmetric carbon atoms), or a halogen, with the proviso that at least one of R¹, R² and R³ is a substituted or unsubstituted five-membered aromatic heterocycle having at least one oxygen or sulfur atom; and where X is selected from nitrogen, oxygen, and sulfur.

In certain cases the substituents R¹, R², and R³, contribute to optical isomerism and/or stereo isomerism. Salts, solvates, hydrates, prodrug forms of the compounds also are possible. All such forms are embraced by the present invention. Thus the compounds of the subject invention include salts, solvates, hydrates, prodrug and isomer forms thereof, including the pharmaceutically acceptable salts, solvates, hydrates, prodrugs and isomers thereof.

Of interest in certain embodiments are substituted isoxazoles, such as a compound of formula (I) where X is oxygen and having a structure of formula (II):

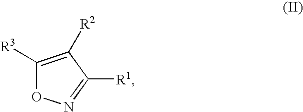

where R¹, R² and R³ are as defined above.

In a related embodiment, the substituted isoxazole of formula (II) is a 3,5-substituted isozazole, where R² is hydrogen, and R¹ and R³ are as defined above. Of specific interest is a 3,5-substituted isozazole of formula (II) in which R² is hydrogen, and R¹ is a substituted or unsubstituted acyl, a substituted or unsubstituted amino, or a substituted or unsubstituted straight-chain or branched C₁-C₂₀-alkyl or C₂-C₂₀-alkenyl (with or without asymmetric carbon atoms), and R³ is a substituted or unsubstituted five-membered aromatic heterocycle having at least one oxygen or sulfur atom. In another related embodiment, the substituted isoxazole compound is a substituted 5-thiophen-2-yl-isoxazole of formula (II), where R¹ and R² are as defined above, and R³ is a substituted or unsubstituted thiophene.

Of special interest are substituted 5-thiophen-2-yl-isoxazole compounds having a structure of formula (III):

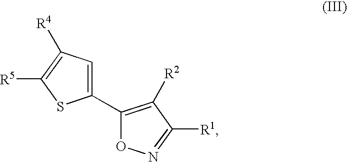

where each R¹ and R² is the same or different and each independently is hydrogen or a substituted or unsubstituted group selected from acyl, acyloxy, aliphatic, alkoxy, amino, cycloalkyl, aryl, aryloxy, heteroaryl or heterocycle, or cyano, with the proviso that at least one of R¹ and R² is other than hydrogen; and R⁴ and R⁵ each independently is hydrogen or a substituted or unsubstituted group selected from acyl, amino, heterocycle, sulfonyl, halogen, and straight-chain or branched C₁-C₁₅-alkyl or C₂-C₁₅-alkenyl or C₁-C₁₅-alkyloxy (with or without asymmetric carbon atoms).

In accordance with compounds of formula (III), R1 may be optionally substituted by one or more substituents. Examples include substituents selected from the group consisting of heteroatom, halogen, cyano, nitro, azide, lower alkyl, lower alkoxy, lower alkoxy substituted by halogen, lower alkyl substituted by halogen, C(O)O-lower alkyl, lower alkylsulfonyl, —NR^{a}R^{b}, —C(O)—NR^{a}R^{b}, —C(O)-heterocycle, benzyloxy, heterocycle optionally substituted by hydroxy, halogen or lower alkyl, and heteroaryl optionally substituted by lower alkyl, where R^{a} and R^{b} are each independently hydrogen, lower alkylsulfonyl, —C(O)H, —(CH₂), —N(R^{c})₂, —(CH₂)ₙ—O-lower alkyl, —(CH₂)ₙ—S-lower alkyl, —(CH₂)ₙ—S(O)₂-lower alkyl, heteroarylsulfonyl, lower alkyl, —(CH₂)ₙ-heterocycle optionally substituted by lower alkyl, —(CH₂)ₙ-cycloalkyl, —(CH₂)ₙ-heteroaryl, —(CH₂)ₙ—OH, or —(CO)—R^{d}, R^{c} is hydrogen or a lower alkyl, R^{d} is lower alkyl, cycloalkyl or heteroaryl, and n is 0, 1 or 2

In certain embodiments, the compound is a substituted 5-thiophen-2-yl-isoxazole of formula (III) in which R¹ is a substituted or unsubstituted acyl, a substituted or unsubstituted amino, a substituted or unsubstituted heterocycle, a substituted or unsubstituted straight-chain or branched C₁-C₂₀-alkyl or C₂-C₂₀-alkenyl (with or without asymmetric carbon atoms), each of R² and R⁴ is hydrogen, and R⁵ is hydrogen, a substituted or unsubstituted acyl, a substituted or unsubstituted amino, a substituted or unsubstituted heterocycle, a substituted or unsubstituted sulfonyl group, a halogen, or a substituted or unsubstituted straight-chain or branched C₁-C₁₅-alkyl or C₂-C₁₅-alkenyl or C₁-C₁₅-alkyloxy (with or without asymmetric carbon atoms).

Compounds in particular embodiments include those where the compound is a substituted 5-thiophen-2-yl-isoxazole of formula (III), where R², R⁴ and R⁵ are each hydrogen, and R¹ is a substituted or unsubstituted acyl, a substituted or unsubstituted amino, a substituted or unsubstituted heterocycle, or a substituted or unsubstituted straight-chain or branched C₁-C₂₀-alkyl or C₂-C₂₀-alkenyl (with or without asymmetric carbon atoms).

In certain embodiments, the compound is a substituted 5-thiophen-2-yl-isoxazole of formula (III) in which R², R⁴ and R⁵ are each hydrogen, and R¹ is a substituted acyl, such as a 5-thiophen-2-yl-isoxazole-3-acyl compound having a structure according to formula (IV):

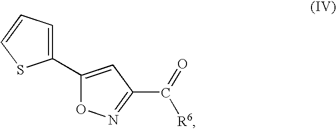

where R⁶ is a substituted or unsubstituted group selected from amino, a straight-chain or branched C₁-C₁₅-alkyl or C₂-C₁₅-alkenyl or C₁-C₁₅-alkyloxy (with or without asymmetric carbon atoms), cycloalkyl, aryl, heteroaryl, heterocycle, and cyano.

In accordance with compounds of formula (IV), the R⁶ group may be optionally substituted with one or more substituents. Examples of R⁶ substituents include, but are not limited to, substituents selected from the group consisting of halogen, cyano, nitro, lower alkyl, lower alkoxy, lower alkoxy substituted by halogen, lower alkyl substituted by halogen, —C(O)O-lower alkyl, lower alkylsulfonyl, —NR^{a}R^{b}, —C(O)—NR^{a}R^{b}, —C(O)-heterocycle, benzyloxy, heterocycle optionally substituted by hydroxy, halogen or lower alkyl, and heteroaryl optionally substituted by lower alkyl, where R^{a} and R^{b} are each independently hydrogen, lower alkylsulfonyl, —C(O)H, —(CH₂), —N(R)₂, —(CH₂)ₙ—O-lower alkyl, —(CH₂)ₙ—S-lower alkyl, —(CH₂)ₙ—S(O)₂-lower alkyl, heteroarylsulfonyl, lower alkyl, —(CH₂)ₙ-heterocycle optionally substituted by lower alkyl, —(CH₂)ₙ-cycloalkyl, —(CH₂)ₙ-heteroaryl, —(CH₂)ₙ—OH, or —(CO)—R′, wherein R′ is lower alkyl, cycloalkyl or heteroaryl, and where n is 0, 1 or 2, and where R is hydrogen or lower alkyl.

In certain embodiments, the compound is a 5-thiophen-2-yl-isoxazole-3-carbonylamino compound of formula (IV), where R⁶ is an amino group that is substituted with a straight- or branched-chain alkyl or alkenyl group containing from one to six carbon atoms which is optionally substituted; or an amino group that is substituted with an aryl, heterocycle, cycloalkyl or heterocycloalkyl group containing from three to six carbon atoms which is optionally substituted. In a specific embodiment, the compound is a 5-thiophen-2-yl-isoxazole-3-carbonylamino compound of formula (IV) in which R⁶ is an amino group that is substituted with a straight- or branched-chain alkyl or alkenyl group containing from one to six carbon atoms which is optionally substituted. In another specific embodiment, the compound is a 5-thiophen-2-yl-isoxazole-3-carbonylamino compound of formula (IV) in which R⁶ is an amino group that is substituted with a straight- or branched-chain alkyl or alkenyl group containing from two to six carbon atoms which is optionally substituted.

Examples of substituted isoxazoles in accordance with one or more of the above structural formulae include, but are not limited to, N-butyl-5-thiophen-2-yl-1,2-oxazole-3-carboxamide; N-ethyl-5-thiophen-2-yl-1,2-oxazole-3-carboxamide; N-propyl-5-thiophen-2-yl-1,2-oxazole-3-carboxamide; N-prop-2-enyl-5-thiophen-2-yl-1,2-oxazole-3-carboxamide; N-butan-2-yl-5-thiophen-2-yl-1,2-oxazole-3-carboxamide; N-propan-2-yl-5-thiophen-2-yl-1,2-oxazole-3-carboxamide; N-(3-diethylaminopropyl)-5-thiophen-2-yl-1,2-oxazole-3-carboxamide; N-(2-methylpropyl)-5-thiophen-2-yl-1,2-oxazole-3-carboxamide; N-(cyclopropylmethyl)-5-thiophen-2-yl-1,2-oxazole-3-carboxamide; N-cyclopropyl-5-thiophen-2-yl-1,2-oxazole-3-carboxamide; N-[2-(cyclopropylamino)-2-oxoethyl]-5-thiophen-2-yl-1,2-oxazole-3-carboxamide; N-prop-2-ynyl-5-thiophen-2-yl-1,2-oxazole-3-carboxamide; N-[2-(tert-butylamino)-2-oxoethyl]-5-thiophen-2-yl-1,2-oxazole-3-carboxamide; N-[(4,6-dimethyl-2-oxo-1H-pyridin-3-yl)methyl]-5-thiophen-2-yl-1,2-oxazole-3-carboxamide; N-cyclohexyl-5-thiophen-2-yl-1,2-oxazole-3-carboxamide; N-cycloheptyl-5-thiophen-2-yl-1,2-oxazole-3-carboxamide; N-cyclopentyl-5-thiophen-2-yl-1,2-oxazole-3-carboxamide; N,N-diethyl-5-thiophen-2-yl-1,2-oxazole-3-carboxamide; N-(1-methylpiperidin-4-yl)-5-thiophen-2-yl-1,2-oxazole-3-carboxamide; 5-propyl-N-(thiophen-2-ylmethyl)-1,2-oxazole-3-carboxamide; N-(2-methoxyethyl)-5-thiophen-2-yl-1,2-oxazole-3-carboxamide; N-(1-methoxypropan-2-yl)-5-thiophen-2-yl-1,2-oxazole-3-carboxamide; 5-thiophen-2-yl-N-(2,2,2-trifluoroethyl)-1,2-oxazole-3-carboxamide; 5-thiophen-2-yl-N-(thiophen-2-ylmethyl)-1,2-oxazole-3-carboxamide; N-(2-dimethylamino-2-thiophen-2-ylethyl)-5-thiophen-2-yl-1,2-oxazole-3-carboxamide; N-(4-methylcyclohexyl)-5-thiophen-2-yl-1,2-oxazole-3-carboxamide; (4-methylpiperazin-1-yl)-(5-thiophen-2-yl-1,2-oxazol-3-yl)methanone; 2-[methyl-[(5-thiophen-2-yl-1,2-oxazol-3-yl)methyl]amino]-N-propan-2-ylacetamide; methyl-[2-oxo-2-(propan-2-ylamino)ethyl]-[(5-thiophen-2-yl-1,2-oxazol-3-yl)methyl]azanium; 2-[ethyl-[(5-thiophen-2-yl-1,2-oxazol-3-yl)methyl]amino]-N-propan-2-ylacetamide; ethyl-[2-oxo-2-(propan-2-ylamino)ethyl]-[(5-thiophen-2-yl-1,2-oxazol-3-yl)methyl]azanium; pyrrolidin-1-yl-(5-thiophen-2-yl-1,2-oxazol-3-yl)methanone; piperidin-1-yl-(5-thiophen-2-yl-1,2-oxazol-3-yl)methanone; 5-methyl-N-(thiophen-2-ylmethyl)-1,2-oxazole-3-carboxamide; N-[2-(benzoylamino)ethyl]-5-thiophen-2-yl-1,2-oxazole-3-carboxamide; (2-methylpiperidin-1-yl)-(5-thiophen-2-yl-1,2-oxazol-3-yl)methanone; azepan-1-yl-(5-thiophen-2-yl-1,2-oxazol-3-yl)methanone; 5-propan-2-yl-N-(thiophen-2-ylmethyl)-1,2-oxazole-3-carboxamide; thiomorpholin-4-yl-(5-thiophen-2-yl-1,2-oxazol-3-yl)methanone; 1-[4-[(5-thiophen-2-yl-1,2-oxazol-3-yl)methyl]piperazin-4-ium-1-yl]ethanone; N-cyclopropyl-2-[methyl-[(5-thiophen-2-yl-1,2-oxazol-3-yl)methyl]amino]acetamide; [2-(cyclopropylamino)-2-oxoethyl]-methyl-[(5-thiophen-2-yl-1,2-oxazol-3-yl)methyl]azanium; [2-(tert-butylamino)-2-oxoethyl]-ethyl-[(5-thiophen-2-yl-1,2-oxazol-3-yl)methyl]azanium; 4-[(5-thiophen-2-yl-1,2-oxazol-3-yl)methyl]piperazin-2-one; 4-[(5-thiophen-2-yl-1,2-oxazol-3-yl)methyl]piperazin-4-ium-2-one; N-ethyl-5-methyl-N-[2-oxo-2-(thiophen-2-ylmethylamino)ethyl]-1,2-oxazole-3-carboxamide; N-(2-phenylethyl)-5-thiophen-2-yl-1,2-oxazole-3-carboxamide; (2-ethylpiperidin-1-yl)-(5-thiophen-2-yl-1,2-oxazol-3-yl)methanone; 5-(2-methylpropyl)-N-(thiophen-2-ylmethyl)-1,2-oxazole-3-carboxamide; methyl 2-[(5-thiophen-2-yl1,2-oxazole-3-carbonyl)amino]acetate; N-(phenylmethyl)-5-thiophen-2-yl-1,2-oxazole-3-carboxamide; N-(1-phenylethyl)-5-thiophen-2-yl-1,2-oxazole-3-carboxamide; (2,6-dimethylpiperidin-1-yl)-(5-thiophen-2-yl-1,2-oxazol-3-yl)methanone; N-ethyl-5-[(thiophen-2-ylmethylamino)methyl]-1,2-oxazole-3-carboxamide; N-methyl-1-(5-thiophen-2-yl-1,2-oxazol-3-yl)methanamine; N-[2-(azepan-1-yl)-2-thiophen-2-ylethyl]-5-thiophen-2-yl-1,2-oxazole-3-carboxamide; N-methyl-N-phenyl-5-thiophen-2-yl-1,2-oxazole-3-carboxamide; N-(2-piperidin-1-yl-2-thiophen-2-ylethyl)-5-thiophen-2-yl-1,2-oxazole-3-carboxamide; (4-methylpiperidin-1-yl)-(5-thiophen-2-yl-1,2-oxazol-3-yl)methanone; N-[2-[(4-fluorophenyl)amino]-2-oxoethyl]-N-methyl-5-thiophen-2-yl-1,2-oxazole-3-carboxamide; N-[(2-fluorophenyl)methyl]-5-thiophen-2-yl-1,2-oxazole-3-carboxamide; N,N-dicyclohexyl-5-thiophen-2-yl-1,2-oxazole-3-carboxamide; N-phenyl-5-thiophen-2-yl-1,2-oxazole-3-carboxamide; 5-cyclopropyl-N-(thiophen-2-ylmethyl)-1,2-oxazole-3-carboxamide; N-tert-butyl-2-[ethyl-[(5-thiophen-2-yl-1,2-oxazol-3-yl)methyl]amino]acetamide; N-(oxolan-2-ylmethyl)-5-thiophen-2-yl-1,2-oxazole-3-carboxamide; N-(2-morpholin-4-ylethyl)-5-thiophen-2-yl-1,2-oxazole-3-carboxamide; N-(3-morpholin-4-ylpropyl)-5-thiophen-2-yl-1,2-oxazole-3-carboxamide; N-[(4-fluorophenyl)methyl]-5-thiophen-2-yl-1,2-oxazole-3-carboxamide; 5-phenyl-N-(thiophen-2-ylmethyl)-1,2-oxazole-3-carboxamide; N-[2-[(3-fluoro-4-methylbenzoyl)amino]ethyl]-5-thiophen-2-yl-1,2-oxazole-3-carboxamide; N-[(4-acetamidophenyl)methyl]-5-thiophen-2-yl-1,2-oxazole-3-carboxamide; N-[2-(4-methylpiperidin-1-yl)-2-thiophen-2-ylethyl]-5-thiophen-2-yl-1,2-oxazole-3-carboxamide; N-(2-dimethylamino-2-thiophen-2-ylethyl)-5-phenyl-1,2-oxazole-3-carboxamide; N-(4-fluorophenyl)-5-thiophen-2-yl-1,2-oxazole-3-carboxamide; N-(2-fluorophenyl)-5-thiophen-2-yl-1,2-oxazole-3-carboxamide; N-(4-acetamidophenyl)-5-thiophen-2-yl-1,2-oxazole-3-carboxamide; 5-(4-fluorophenyl)-N-(thiophen-2-ylmethyl)-1,2-oxazole-3-carboxamide; N-(2-methylsulfanylphenyl)-5-thiophen-2-yl-1,2-oxazole-3-carboxamide; 5-[(3-cyclopentylpropanoyl-(thiophen-2-ylmethyl)amino)methyl]-N-(2-methylpropyl)-1,2-oxazole-3-carboxamide; N-(2-methylpropyl)-5-[(thiophen-2-ylmethylamino)methyl]-1,2-oxazole-3-carboxamide; N-ethyl-5-[(pentan-3-yl-(thiophene-2-carbonyl)amino)methyl]-1,2-oxazole-3-carboxamide; ethyl 4-(5-thiophen-2-yl1,2-oxazole-3-carbonyl)piperazine-1-carboxylate; N-[3-(methylcarbamoyl)phenyl]-5-thiophen-2-yl-1,2-oxazole-3-carboxamide; N-(3-acetamidophenyl)-5-thiophen-2-yl-1,2-oxazole-3-carboxamide; N-[2-[(5Z)-2,4-dioxo-5-(thiophen-2-ylmethylidene)-1,3-thiazolidin-3-yl]ethyl]-5-methyl-1,2-oxazole-3-carboxamide; N-(2,4-difluorophenyl)-5-thiophen-2-yl-1,2-oxazole-3-carboxamide; N-(2,5-difluorophenyl)-5-thiophen-2-yl-1,2-oxazole-3-carboxamide; N-cyclopropyl-2-[4-[(5-thiophen-2-yl-1,2-oxazol-3-yl)methyl]piperazin-1-yl]acetamide; 1-[4-[(5-thiophen-2-yl-1,2-oxazol-3-yl)methyl]piperazin-1-yl]ethanone; 2-[ethyl-[(5-phenyl-1,2-oxazol-3-yl)methyl]amino]-N-(thiophen-2-ylmethyl)acetamide; ethyl-[2-oxo-2-(thiophen-2-ylmethylamino)ethyl]-[(5-phenyl-1,2-oxazol-3-yl)methyl]azanium; N-ethyl-N-[(4-methyl-1,2,5-oxadiazol-3-yl)methyl]-5-thiophen-2-yl-1,2-oxazole-3-carboxamide; morpholin-4-yl-(5-thiophen-2-yl-1,2-oxazol-3-yl)methanone; 5-(4-methylphenyl)-N-(thiophen-2-ylmethyl)-1,2-oxazole-3-carboxamide; methyl 3-amino-5-(3-phenyl-1,2-oxazol-5-yl)thiophene-2-carboxylate; 2-[[4-[5-(5-ethylthiophen-2-yl)-1,2-oxazol-3-yl]phenyl]sulfonylamino]-3-methylbutanoic acid; 3-methyl-2-[[4-[5-(5-methylthiophen-2-yl)-1,2-oxazol-3-yl]phenyl]sulfonylamino]butanoic acid; (2R)-3-methyl-2-[[4-[5-(5-methylthiophen-2-yl)-1,2-oxazol-3-yl]phenyl]sulfonylamino]butanoic acid; [3-methyl-5-(5-methylthiophen-2-yl)-1,2-oxazol-4-yl]carbamate; 5-(3-methyl-1,2-oxazol-5-yl)-N-[[5-oxo-1-[4-(2-oxopiperidin-1-yl)phenyl]pyrrolidin-3-yl]methyl]thiophene-2-carboxamide; 2-[2-methyl-5-(3-methyl-1,2-oxazol-5-yl)thiophen-3-yl]sulfonyl-3,4-dihydro-1H-isoquinoline; 2-[5-(3,4-dimethyl-1,2-oxazol-5-yl)-2-methylthiophen-3-yl]sulfonyl-3,4-dihydro-1H-isoquinoline; 1-(2-chlorophenyl)ethyl N-[3-methyl-5-(5-methylthiophen-2-yl)-1,2-oxazol-4-yl]carbamate; 3-(5-heptylthiophen-2-yl)-5-(5-propylthiophen-2-yl)-1,2-oxazole; 3-(4-octoxyphenyl)-5-[5-[(E)-pent-1-enyl]thiophen-2-yl]-1,2-oxazole; 5-(5-butylthiophen-2-yl)-3-(4-octylphenyl)-1,2-oxazole; 5-(5-butylthiophen-2-yl)-3-(4-octoxyphenyl)-1,2-oxazole; 3-(5-butylthiophen-2-yl)-5-(5-nonylthiophen-2-yl)-1,2-oxazole; 3-(5-decylthiophen-2-yl)-5-(5-pentylthiophen-2-yl)-1,2-oxazole; 3-(5-heptylthiophen-2-yl)-5-(5-nonylthiophen-2-yl)-1,2-oxazole; and 3-(5-heptylthiophen-2-yl)-5-(5-pentylthiophen-2-yl)-1,2-oxazole.

In certain embodiments, the compound is a substituted 5-thiophen-2-yl-isoxazole-3-carboxylic acid propylamide (also referred to as N-propyl-5-(2-thienyl)isoxazole-3-carboxamide), which has the following structure:

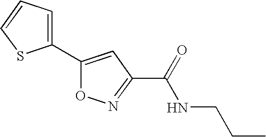

Thus, the compounds of certain embodiments of the invention range in molecular weight from about 100 to about 700 daltons, including from about 125 to about 600 daltons such as from about 150 to about 450. Compounds of the invention may contain from about 4 to about 50 carbon atoms and contain at least one other type of atom, including but not limited to nitrogen, oxygen, sulfur, bromine, fluorine, and/or chlorine atoms. As discussed above, the non-carbon atoms can be present as part of an aromatic ring structure, a substituent of the aromatic ring group, as part of a non-aromatic ring structure, or as another structural element.

Also of particular interest are analogues/derivatives of the above compounds, where such analogues/derivatives have modulatory effects on the interaction of transcriptional repressor complexes to a Site C site in the TERT promoter such that telomerase expression is increased in cells when the compounds are administered according to the methods of the subject invention.

### Methods of Use

The compounds of the invention can increase TERT expression in a target cell population when contacted thereto. In general, the cells of interest are contacted to a composition containing an effective amount of at least one of the compounds of the subject invention in an acceptable form (e.g., an acceptable carrier). By effective amount is meant that the compound (or compounds) of the invention are present in a quantity that can increase the expression of TERT in the target cells. In practicing these methods, the cells of interest may be contacted to a compound or compounds of the invention in an in vitro or ex vivo culture system or in vivo. For example, a compound(s) of the invention may be contacted to primary cells grown under standard tissue culture conditions or alternatively a compound(s) of the invention can be contacted to cells that are part of a whole animal (e.g., administered to a subject).

The invention also includes a method of screening compounds of the invention for inhibiting binding of a transcriptional repressor protein/protein complex to a TERT promoter that comprises at least one of Site C binding site. In general, this method involves determining whether a substituted azole compound is capable of inhibiting binding of the transcriptional repressor protein/protein complex to the Site C binding site. In a related embodiment, the invention also includes a method of screening compounds of the invention for increasing TERT expression in a cell containing a TERT expression system that comprises at least one Site C binding site in its promoter. This method comprises (i) contacting the cell with an effective amount of a substituted azole compound, and (ii) determining whether the compound inhibits binding of a transcriptional repressor protein/protein complex to the Site C binding site.

The determining step in the subject screening methods of the invention can be carried out by any one or more of a variety a protocols for characterizing TERT expression and/or the inhibition of binding of the transcriptional repressor protein/protein complex to the Site C binding site of the TERT expression system. For example, screening may be a reconstitution assay, cell-based assay, enzyme assay, ELISA assay or other related biological assay for assessing TERT expression and/or the inhibition of binding of the transcriptional repressor protein/protein complex to the Site C binding site of the TERT expression system, and the determining or assessment step suitable for application in such assays are well known and involve routine protocols. Screening may also include in silico approaches, in which one or more physical and/or chemical attributes of a compound of interest are expressed in a computer-readable format and evaluated by any one or more of a variety molecular modeling and/or analysis programs and algorithms suitable for this purpose.

Thus the screening methods of the invention can be carried out in vitro or in vivo. For example, when the TERT promoter is in a cell, the cell may be in vitro or in vivo, and the determining of whether the compound is capable of inhibiting binding comprises (i) contacting the cell with an effective amount of the substituted azole compound, and (ii) assessing whether said substituted azole compound inhibits binding of the transcriptional repressor protein/protein complex to the Site C binding site. In certain embodiments, inhibition of binding of the transcriptional repressor protein/protein complex to the Site C binding site increases the proliferative capacity of the cell. In some embodiments, inhibition of binding of the transcriptional repressor protein/protein complex to the Site C binding site delays the senescence of the cell. In yet additional embodiments, the substituted azole inhibits binding of the transcriptional repressor protein/protein complex to the Site C binding site. As such, determining whether a substituted azole compound is capable of inhibiting binding of the transcriptional repressor protein/protein complex to the Site C binding site may be carried out by any number of methods, as well as combinations thereof.

In certain embodiments, the screening is or comprises part of an assay selected from a potency assay, a compound or product release assay, and combinations thereof. The potency assay characterizes one or more biological activities of a compound of interest, where biological activity is characterized in general by TERT expression levels and/or inhibiting binding of the transcriptional repressor protein/protein complex to the Site C binding site of a TERT expression system. Such a potency assay may also be exploited in the development and/or validation of assays, as well as for a compound release assay. The compound release assay involves assessment of one or more of sterility, safety, purity, identity and potency of a compound of interest.

Thus in some embodiments, when the screening method employs a substituted azole that inhibits binding of the transcriptional repressor protein/protein complex to the Site C binding site, the substituted azole may be comprised as a pharmaceutical composition of the invention. In certain embodiments, the screening is a release assay for the pharmaceutical composition. In some embodiments, the screening is a potency assay for the pharmaceutical composition.

Accordingly, in certain embodiments, the screening methods of the invention are carried out for product release, such as to demonstrate and/or confirm that a compound, such as a pharmaceutical composition comprising the compound, is one or more of safe, pure, potent, effective and stable. As such, the screening methods of the invention may include demonstration of manufacturing and product consistency, including characterization for product release involving assessment of one or more of sterility, safety, purity, identity and potency.

Of specific interest are screening methods of the invention that assess potency of a substituted azole compound of interest. By “potency” is intended the specific ability or capacity of a compound to effect a given result. In general, tests for potency consist of either in vitro or in vivo tests, or both, which have been specifically adapted for each product so as to indicate its potency. Thus, potency assays indicate biological activity(s) specific/relevant to the product of interest. As noted above, the potency assays of the invention generally involve the generation of data regarding TERT expression and/or inhibition of binding of the transcriptional repressor protein/protein complex to the Site C binding site. Such data may include, but is not limited to, qualitative and/or quantitative results for compound activity, lot release, predefined acceptance and/or rejection criteria (demonstrate lot to lot consistency), include appropriate reference material/controls, be validated for licensure, measure activity of one or more components that may be necessary for product activity, and/or indicate product stability.

Potency measurements can be direct (e.g., biological assay) or indirect (e.g., surrogate assay(s) correlated to biological activity that may include one of many assays that measure product quality). For example, potency can be measured by simple identity markers that exhibit minimal variability from assay to assay over time, including functional biomarkers that correlate with cellular differentiation and senescence. This includes measurement of one or more of cellular proliferation, cellular survival, and/or senescence, as well as biomarkers from analytic, genomic and/or proteomic-based techniques that correlate to the biological activity of interest. For instance, determining expression of TERT and/or inhibition of binding of the transcriptional repressor protein/protein complex to the Site C binding site can include various approaches for indirect potency measurements, including analytical assays such as a non-bioassay method correlated to a unique and/or specific activity of the compound (e.g., immunochemical procedures such as ELISA, ELISPOT, Q-flow cytometry, quantitative western blots; and molecular and biochemical procedures such as enzymatic assays, Q-PCR, RT-PCR, microarray/genomics, proteomics).

Thus potency measurement may be carried out in vivo in animal models or from clinical data (e.g., assessment of gene function, cell survival and so forth), and in vitro such as in cell and/or tissue culture (e.g., assessment of signaling pathways, proliferation, enzymatic activity, cell survival and so forth).

### Utility

The compounds of the invention find use in a variety of applications. For example, compounds of the invention find use in applications for increasing the proliferative capacity of cells grown in vitro (e.g., immortalizing cells). As such, the compounds of the invention find use in expanding cells for a variety uses, including expanding cells for use in diagnostic assays, expanding cells for use in preparative protocols (e.g., expanding antibody-producing cells of cells expressing a protein/factor of interest), expanding cells to facilitate studying the cells themselves (e.g., expanding rare stem cells harvested from a subject). In addition, the compounds of the invention can be used to expand cells that will themselves be administered to a subject for experimental or therapeutic purposes, for example in expanding cells for genetic alteration (e.g., gene therapeutic purposes). As such, the compounds and methods of the subject invention are useful in any application in which an increase in cellular proliferation or a reduction in cellular senescence is advantageous.

For example, the screening methods of the invention find use in a variety of applications, including identifying and/or testing compounds of the invention for use in a wide range of research and therapeutic applications, such as pharmaceutical development, manufacturing, and quality assurance/control, as well as immortalization of cell lines and treating conditions in a subject characterized by cellular senescence. This includes use of the screening methods of the invention for performing research, as well as for pharmaceutical compliance related to GLP (“Good Laboratory Practice”) and GMP (“Good Manufacturing Practice” also referred to as “cGMP” or “current Good Manufacturing Practice”)) and laboratory services. Thus the screening methods of the invention find broad use in research and lead development, sample analysis, as well as assay development, validation, drug regulatory submissions and compliance for new drug substances and drug products, drug product release and compound auditing in general. By “compound auditing” is intended quality assurance and/or quality control of a compound.

Compound auditing in accordance with the subject screening methods may be exploited in multiple settings. One example is in assay development or simply to transfer an assay from one location to another, whether or not it requires GLP and/or GMP compliance. This aspect generally involves use of the subject screening methods of the invention to ensure that a compound of interest performs consistently and provides continuity in an assay over time. Statistical data analysis and related relevant data analysis tools can be exploited to best match the compound and use of interest. For instance, the screening method can be performed under “research level” protocols to identify those parameters such as the limit of detection (LOD), the limit of quantitation (LOQ) and the linear range necessary for assay validation and/or transfer. As such, the screening methods find use in compiling and executing SOPs (“Standard Operating Procedure” or “Standard Operating Protocol”) which can be used for compound auditing.

Additional uses of the screening methods of the invention include the generation and/or execution one or more GLP or GMP protocols that assess one or more of linearity, accuracy, precision, specificity, robustness, ruggedness and system suitability for one or more compounds of interest for a given end use. This includes assays for identifying as well as testing of a compound of interest, including QA and/or QC, as well as generating controls that may be aliquoted under GLP or GMP compliance which may be used over several years depending upon the stability of the compound of interest.

The subject screening methods of the invention find particular use in qualitative and/or quantitative potency assays for routine lot release, lot comparisons, sampling, and stability assessment of a compound of interest.

The screening methods of the invention may also be used in a multiple assay approach (i.e., assay matrix), such as when it is desirable to develop or use more than a single assay (e.g., an assay matrix often finds use when there is limited knowledge of product and mechanism of action, the product has multiple components with multiple biological activities, time is constrained due to limited product stability, biological assay is not quantitative and the like). Thus the subject screening methods may find use in a combination of assays where the combined results, constitute an acceptable product release and/or potency assay (e.g. a quantitative physical assay along with a qualitative bioassay).

The compounds of the invention find use in a variety of therapeutic applications. In general, therapeutic applications of interest are those in which reduced activity or expression of TERT (or shortened telomeres) is the cause or a compounding factor in disease progression. In such cases, administering to a subject a compound of the invention in an effective amount will ameliorate the symptoms of the disease or deleterious condition. By effective amount is meant a dosage sufficient to increase TERT expression in the target cell(s), as desired. A variety of conditions associated with low activity of TERT can be treated with the compounds of the present invention including, but not limited to, progeria, atherosclerosis, cardiovascular diseases, osteoarthritis, osteoporosis, Alzheimer's disease, macular degeneration, liver cirrhosis, rheumatoid arthritis, and AIDS or HIV infection. In addition, representative compounds of the invention can increase the life span of an animal.

The subject compounds find use in the treatment of a variety of different conditions in which the enhancement of TERT expression in the host is desired. By treatment is meant that at least an amelioration of the symptoms associated with the condition afflicting the host is achieved, where amelioration is used in a broad sense to refer to at least a reduction in the magnitude of a parameter, e.g. symptom (such as inflammation), associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

As indicated above, the subject invention provides compounds and methods of treating disease conditions resulting from a lack of, or reduced, TERT expression. Representative disease conditions for each category are now described in greater detail separately.

One representative disease condition that may be treated according to the subject invention is Progeria, or Hutchinson-Gilford syndrome. This condition is a disease of shortened telomeres for which no known cure exists. It afflicts children, who seldom live past their early twenties. In many ways progeria parallels aging itself. However, these children are born with short telomeres. Their telomeres do not shorten at a faster rate; they are just short to begin with. The subject methods can be used in such conditions to further delay natural telomeric shortening and/or increase telomeric length, thereby treating this condition.

Another specific disease condition in which the subject compounds and methods find use is in immune senescence. The effectiveness of the immune system decreases with age. Part of this decline is due to fewer T-lymphocytes in the system, a result of lost replicative capacity. Many of the remaining T-lymphocytes experience loss of function as their telomeres shorten and they approach senescence. The subject methods can be employed to inhibit immune senescence due to telomere loss. Because hosts with aging immune systems are at greater risk of developing pneumonia, cellulitis, influenza, and many other infections, the subject methods reduce morbidity and mortality due to infections.

The subject invention also finds use in AIDS therapy. HIV, the virus that causes AIDS, invades white blood cells, particularly CD4 lymphocyte cells, and causes them to reproduce high numbers of the HIV virus, ultimately killing cells. In response to the loss of immune cells (typically about a billion per day), the body produces more CD8 cells to be able to suppress infection. This rapid cell division accelerates telomere shortening, ultimately hastening immune senescence of the CD8 cells. Anti-retroviral therapies have successfully restored the immune systems of AIDS patients, but survival depends upon the remaining fraction of the patient's aged T-cells. Once shortened, telomere length has not been naturally restored within cells. The subject methods can be employed to restore this length and/or prevent further shortening. As such the subject methods can spare telomeres and is useful in conjunction with the anti-retroviral treatments currently available for HIV.

Yet another type of disease condition in which the subject invention finds use is cardiovascular disease. The compounds of the invention can be employed to extend telomere length and replicative capacity of endothelial cells lining blood vessel walls (DeBono, Heart 80:110-1, 1998). Endothelial cells form the inner lining of blood vessels and divide and replace themselves in response to stress. Stresses include high blood pressure, excess cholesterol, inflammation, and flow stresses at forks in vessels. As endothelial cells age and can no longer divide sufficiently to replace lost cells, areas under the endothelial layer become exposed. Exposure of the underlying vessel wall increases inflammation, the growth of smooth muscle cells, and the deposition of cholesterol. As a result, the vessel narrows and becomes scarred and irregular, which contributes to even more stress on the vessel (Cooper, Cooke and Dzau, J Gerontol Biol Sci 49: 191-6, 1994). Aging endothelial cells also produce altered amounts of trophic factors (hormones that affect the activity of neighboring cells). These too contribute to increased clotting, proliferation of smooth muscle cells, invasion by white blood cells, accumulation of cholesterol, and other changes, many of which lead to plaque formation and clinical cardiovascular disease (Ibid.). By extending endothelial cell telomeres, the subject methods can be employed to combat the stresses contributing to vessel disease. Many heart attacks may be prevented if endothelial cells were enabled to continue to divide normally and better maintain cardiac vessels. The occurrence of strokes caused by the aging of brain blood vessels may also be significantly reduced by employing the subject methods to help endothelial cells in the brain blood vessels to continue to divide and perform their intended function.

The subject compounds also find use in skin rejuvenation. The skin is the first line of defense of the immune system and shows the most visible signs of aging (West, Arch Dermatol 130(1):87-95, 1994). As skin ages, it thins, develops wrinkles, discolors, and heals poorly. Skin cells divide quickly in response to stress and trauma; but, over time, there are fewer and fewer actively dividing skin cells. Compounding the loss of replicative capacity in aging skin is a corresponding loss of support tissues. The number of blood vessels in the skin decreases with age, reducing the nutrients that reach the skin. Also, aged immune cells less effectively fight infection. Nerve cells have fewer branches, slowing the response to pain and increasing the chance of trauma. In aged skin, there are also fewer fat cells, increasing susceptibility to cold and temperature changes. Old skin cells respond more slowly and less accurately to external signals. They produce less vitamin D, collagen, and elastin, allowing the extracellular matrix to deteriorate. As skin thins and loses pigment with age, more ultraviolet light penetrates and damages skin. To repair the increasing ultraviolet damage, skin cells need to divide to replace damaged cells, but aged skin cells have shorter telomeres and are less capable of dividing (Fossel, REVERSING HUMAN AGING. William Morrow & Company, New York City, 1996).

By practicing the subject methods, e.g., via administration of a compound of the invention topically, one can extend telomere length, and slow the downward spiral that skin experiences with age. Such a product not only helps protect a person against the impairments of aging skin; it also permits rejuvenated skin cells to restore youthful immune resistance and appearance. The subject compounds and methods can be used for both medical and cosmetic skin rejuvenation applications.

Yet another disease condition in which the subject compounds find use in the treatment of osteoporosis. Two types of cells interplay in osteoporosis: osteoblasts make bone and osteoclasts destroy it. Normally, the two are in balance and maintain a constant turnover of highly structured bone. In youth, bones are resilient, harder to break, and heal quickly. In old age, bones are brittle, break easily, and heal slowly and often improperly. Bone loss has been postulated to occur because aged osteoblasts, having lost much of their replicative capacity, cannot continue to divide at the rate necessary to maintain balance (Hazzard et al. PRINCIPLES OF GERIATRIC MEDICINE AND GERONTOLOGY, 2d ed. McGraw-Hill, New York City, 1994). The subject compounds can be employed to lengthen telomeres of osteoblast and osteoclast stem cells, thereby encouraging bone replacement and proper remodeling and reinforcement. The resultant stronger bone improves the quality of life for the many sufferers of osteoporosis and provides savings from fewer fracture treatments. The subject compounds and methods are generally part of a comprehensive treatment regime that also includes calcium, estrogen, and exercise.

Additional disease conditions in which the subject methods find use are described in WO 99/35243, the disclosures of which are herein incorporated by reference.

In addition to the above-described uses, the subject compounds can also be used to extend the lifespan of a mammal. By extend the lifespan is meant to increase the time during which the animal is alive, where the increase is generally 1% or more, such as 5% or more and including 10% or more as compared to a control.

As indicated above, instead of a multicellular animal, the target may be a cell or population of cells which are treated according to the subject methods and then introduced into a multicellular organism for therapeutic effect. For example, the subject compounds may be employed in bone marrow transplants for the treatment of cancer and skin grafts for burn victims. In these cases, cells are isolated from a human donor and then cultured for transplantation back into human recipients. During the cell culturing, the cells normally age and senesce, decreasing their useful lifespans. Bone marrow cells, for instance, lose approximately 40% of their replicative capacity during culturing. This problem is aggravated when the cells are first genetically engineered (Decary, Mouly et al. Hum Gene Ther 7(11): 1347-50, 1996). In such cases, the therapeutic cells must be expanded from a single engineered cell. By the time there are sufficient cells for transplantation, the cells have undergone the equivalent of 50 years of aging (Decary, Mouly et al. Hum Gene Ther 8(12): 1429-38, 1997). Use of the subject compounds spares the replicative capacity of bone marrow cells and skin cells during culturing and expansion and thus significantly improves the survival and effectiveness of bone marrow and skin cell transplants. Any transplantation technology requiring cell culturing can benefit from the subject methods, including ex vivo gene therapy applications in which cells are cultured outside of the animal and then administered to the animal, as described in U.S. Pat. Nos. 6,068,837; 6,027,488; 5,824,655; 5,821,235; 5,770,580; 5,756,283; 5,665,350; the disclosures of which are herein incorporated by reference.

### Pharmaceutical Preparations

A variety of suitable methods of administering a formulation of the present invention to a subject or host, e.g., patient, in need thereof, are available, and, although more than one route can be used to administer a particular formulation, a particular route can provide a more immediate and more effective reaction than another route. Pharmaceutically acceptable excipients are also well-known to those who are skilled in the art, and are readily available. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting.

Formulations suitable for oral administration can consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The subject formulations of the present invention can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are known in the art to be appropriate.

Suppository formulations are also provided by mixing with a variety of bases such as emulsifying bases or water-soluble bases. Formulations suitable for vaginal administration may be presented as pessaries, tampons, creams, gels, pastes, foams.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Dose levels can vary as a function of the specific compound, the nature of the delivery vehicle, and the like. Preferred dosages for a given compound are readily determinable by those of skill in the art by a variety of means.

The dose administered to an animal, particularly a human, in the context of the present invention should be sufficient to effect a prophylactic or therapeutic response in the animal over a reasonable time frame. One skilled in the art will recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to anticancer or immunosuppressive agents that are known to effect the desired growth inhibitory or immunosuppressive response. In the treatment of some individuals with the compounds of the present invention, it may be desirable to use a high dose regimen in conjunction with a rescue agent for non-malignant cells. In such treatment, any agent capable of rescue of non-malignant cells can be employed, such as citrovorum factor, folate derivatives, or leucovorin. Such rescue agents are well known to those of ordinary skill in the art. A rescue agent is preferred which does not interfere with the ability of the present inventive compounds to modulate cellular function.

### Systems & Kits

Systems and Kits with formulations used in the subject methods, are provided. Conveniently, the formulations may be provided in a unit dosage format, which formats are known in the art.

In such systems and kits, in addition to the containers containing the formulation(s), e.g. unit doses, is an informational package insert describing the use of the subject formulations in the methods of the subject invention, e.g., instructions for using the subject unit doses to treat cellular proliferative disease conditions.

These instructions may be present in the subject systems and kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

Compound:

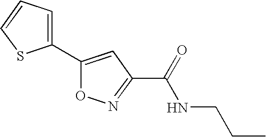

is tested using the assay described in Published United States Patent Application Publication No. US-2006-0199171-A1, the disclosure of which assay is herein incorporated by reference. The compound turned on human TERT expression in this assay.

It is evident from the above results and discussion that the subject invention provides methods of enhancing TERT expression in a cellular or animal host, which methods find use in a variety of applications, including the production of scientific research reagents and therapeutic treatment applications. Accordingly, the subject invention represents significant contribution to the art.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for increasing telomerase reverse transcriptase (TERT) expression in a cell containing a TERT expression system that comprises at least one Site C binding site in its promoter, said method comprising:
contacting said cell with an effective amount of a substituted isoxazole compound that inhibits binding of a transcriptional repressor protein/protein complex to said Site C binding site.

2. The method according to claim 1, wherein said compound has the following structure:

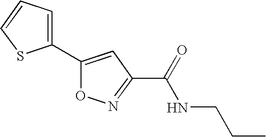

3. The method according to claim 1, wherein the proliferative capacity of said cell is increased.

4. The method according to claim 1, wherein senescence in said cell is delayed.

5. A method for extending the lifespan of a mammal, said method comprising:
administering to said mammal an effective amount of a substituted isoxazole compound that increases TERT expression by inhibiting binding of a transcriptional repressor protein/protein complex to Site C.

6. The method according to claim 5, wherein said mammal is a human.

7. The method according to claim 5, wherein said compound has the following structural formula:

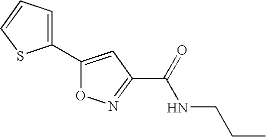

8. A pharmaceutical preparation for increasing the expression of TERT in a subject comprising a substituted isoxazole compound that inhibits binding of a transcriptional repressor protein/protein complex to Site C.

9. The pharmaceutical preparation according to claim 8, wherein said compound has the following structural formula:

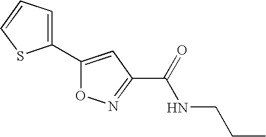

10. A method of screening a compound for inhibiting binding of a transcriptional repressor protein/protein complex to a telomerase reverse transcriptase (TERT) promoter that comprises at least one of Site C binding site, said method comprising:
determining whether a substituted azole compound is capable of inhibiting binding of said transcriptional repressor protein/protein complex to said Site C binding site.

11. The method of claim 10, wherein said TERT promoter is in a cell, and said determining comprises contacting said cell with an effective amount of said substituted azole compound and assessing whether said substituted azole compound inhibits binding of said transcriptional repressor protein/protein complex to said Site C binding site.

12. The method according to claim 10, wherein said substituted azole compound has the following structure: 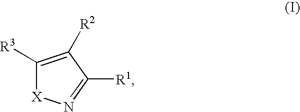
where each R¹, R² and R³ is the same or different and each independently comprises hydrogen or a residue of a group selected from a substituted or unsubstituted acyl, a substituted or unsubstituted amino, a substituted or unsubstituted heterocyle, a substituted or unsubstituted straight-chain or branched C₁-C₂₀-alkyl or C₂-C₂₀-alkenyl (with or without asymmetric carbon atoms), or a halogen, with the proviso that at least one of R¹, R² and R³ is a substituted or unsubstituted five-membered aromatic heterocycle having at least one oxygen or sulfur atom; and where X is selected from nitrogen, oxygen, and sulfur.

13. The method according to claim 11, wherein inhibition of binding of said transcriptional repressor protein/protein complex to said Site C binding site increases the proliferative capacity of said cell.

14. The method according to claim 11, wherein inhibition of binding of said transcriptional repressor protein/protein complex to said Site C binding site delays the senescence of said cell.

15. The method according to claim 10, wherein said substituted azole inhibits binding of said transcriptional repressor protein/protein complex to said Site C binding site.

16. The method according to claim 15, wherein said substituted azole is a 5-thiophen-2-yl-isoxazole-3-carboxylic acid propylamide having the following structural formula:

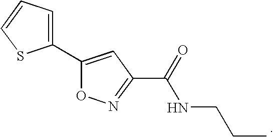

17. The method according to claim 16, wherein said 5-thiophen-2-yl-isoxazole-3-carboxylic acid propylamide is comprised as a pharmaceutical composition.

18. The method according to claim 17, wherein said screening is a release assay for said pharmaceutical composition.

19. The method according to claim 17, wherein said screening is a potency assay for said pharmaceutical composition.


SEQUENCE LISTING (4 sequences)

SEQ ID NO: 1 - DNA, 41 bp, human
ggccccgccctctcctcgcggcgcgagtttcaggcagcgct

SEQ ID NO: 2 - DNA, 13 bp, human
ggcgcgagtttca

SEQ ID NO: 3 - DNA, 10 bp, human
cgcgagtttc

SEQ ID NO: 4 - DNA, 21 bp, human
ggcgcgagtttcaggcagcgc